(19) United States
(12) Patent Application Publication
(10) Pub. No.: US 2013/0335544 A1
(43) Pub. Date: Dec. 19, 2013

(54) ENDOSCOPIC SYSTEM

(71) Applicant: CANON KABUSHIKI KAISHA, Tokyo (JP)

(72) Inventor: Minoru Ookoba, Sagamihara-shi (JP)

(21) Appl. No.: 13/906,536

(22) Filed: May 31, 2013

(30) Foreign Application Priority Data
Jun. 15, 2012 (JP) 2012-135579
Mar. 21, 2013 (JP) 2013-058690

(51) Int. Cl. A61B 1/06

(52) U.S. Cl. CPC A61B 1/06
USPC 348/68

(57) ABSTRACT

An endoscopic system includes an endoscope including: a light source of illumination light that illuminates inside of a test subject; an illumination light exit, from which the illumination light is emitted; and an objective optical system configured to form an image of the inside of the test body illuminated by the illumination light. The endoscopic system further includes: regular reflection light detection means configured to detect blown-off highlights in an image formed by the objective optical system; and illuminance distribution changing means configured to change illuminance distribution of the illumination light when blown-off highlights are detected. The endoscopic system can reduce interference by blown-off highlights due to regular reflection during observation with the endoscope.

### BACKGROUND OF THE INVENTION

1. Field of the Invention

The present invention relates to endoscopic systems.

2. Description of the Related Art

Conventionally endoscopes have been widely available as means to observe inside of a living body and a gap in a narrow space. An endoscope includes an insertion section to be inserted into an observation target, the insertion section being provided with: an illumination light exit, from which illumination light is emitted; and an image forming element that forms an image of diffuse reflected light that is reflected light of the illumination light. An electronic endoscope that is widely in practical use enables observation on a TV monitor, for example, of an image formed by an image-forming element such as a CCD.

Some observation targets may give regular reflection of illumination light, and the reflected light may be directly incident on a light-receiving surface of an image-forming element. Such regular reflected light has high brightness, which may be close to or may exceed the upper limit of a dynamic range of a CCD. Especially during observation of a biological tissue using an endoscope, regular reflection often occurs at mucous membrane or blood covering the biological tissue. The regular reflected light that is direct incident on the light-receiving surface causes a phenomenon where the whole regular-reflection region is displayed in white on the screen, i.e., so-called “blown-out highlights”. Such a phenomenon causes a failure to display information on the blown-out highlights region on the screen, and further interferes with observation at other regions. To prevent this, research has been conducted to remove such blown-out highlights due to regular reflected light.

Specifically Japanese Patent Application Laid-Open No. 2001-224549 describes a method of forming an image free from blown-out highlights due to regular reflection by forming two images by a plurality of pieces of illumination lights and averaging the two images. Japanese Patent Application Laid-Open No. 2009-276545 describes a method of providing illumination as an accessory of an endoscope main body with a mechanism to change the position of the illumination based on an algorithm to change the physical location of the illumination light so as not to cause regular reflection.

### SUMMARY OF THE INVENTION

The endoscopic system described in Japanese Patent Application Laid-Open No. 2001-224549, however, has to keep changing the illumination to keep acquiring two pieces of image information, and has to keep regenerating an image continuously for averaging of images. This means high electric power consumption for operations and a more expensive system.

The endoscopic system described in Japanese Patent Application Laid-Open No. 2009-276545 requires the movement of the illumination position when the system detects blown-out highlights in a captured image. Since this method changes the irradiation direction of illumination light, a part to be observed other than the blown-out highlights may get dark, thus it is difficult to observe the whole area unfortunately.

It is an object of the present invention to provide an endoscopic system capable of reducing blown-out highlights due to regular reflection or the like while avoiding the aforementioned problems.

An endoscopic system of the present invention includes an endoscope including: a light source of illumination light that illuminates inside of a test subject; an illumination light exit, from which the illumination light is emitted; and an objective optical system configured to form an image of the inside of the test body illuminated by the illumination light. The endoscopic system further includes: regular reflection light detection means configured to detect blown-off highlights in an image formed by the objective optical system; and illuminance distribution changing means configured to change illuminance distribution of the illumination light when blown-off highlights are detected.

Further features of the present invention will become apparent from the following description of exemplary embodiments with reference to the attached drawings.

### BRIEF DESCRIPTION OF THE DRAWINGS

FIG. 1 shows the configuration of an endoscopic system of the present invention.

FIGS. 2A, 2B and 2C schematically show the illumination light exit and its illumination state at a distal end of an endoscope.

FIG. 3 is a flowchart to change the illuminance distribution of the present invention.

FIGS. 4A, 4B and 4C are graphs showing the number of pixels versus signal intensity of Example 1.

FIGS. 5A and 5B are graphs showing the number of pixels versus signal intensity of Comparative Example 1.

FIG. 6 shows the distal end of a stereoscopic endoscope in Example 2 and Comparative Example 2.

FIG. 7 shows the configuration of an endoscopic system of the present invention including a memory.

### DESCRIPTION OF THE EMBODIMENTS

Preferred embodiments of the present invention will now be described in detail in accordance with the accompanying drawings.

An endoscopic system of the present invention includes an endoscope including: an illumination light exit, from which illumination light that illuminates inside of a test subject is emitted; and an objective optical system configured to form an image of the inside of the test body illuminated by the illumination light. The endoscopic system further includes: regular reflection light detection means configured to detect blown-off highlights in an image formed by the objective optical system; and illuminance distribution changing means configured to change illuminance distribution of the illumination light when blown-off highlights are detected.

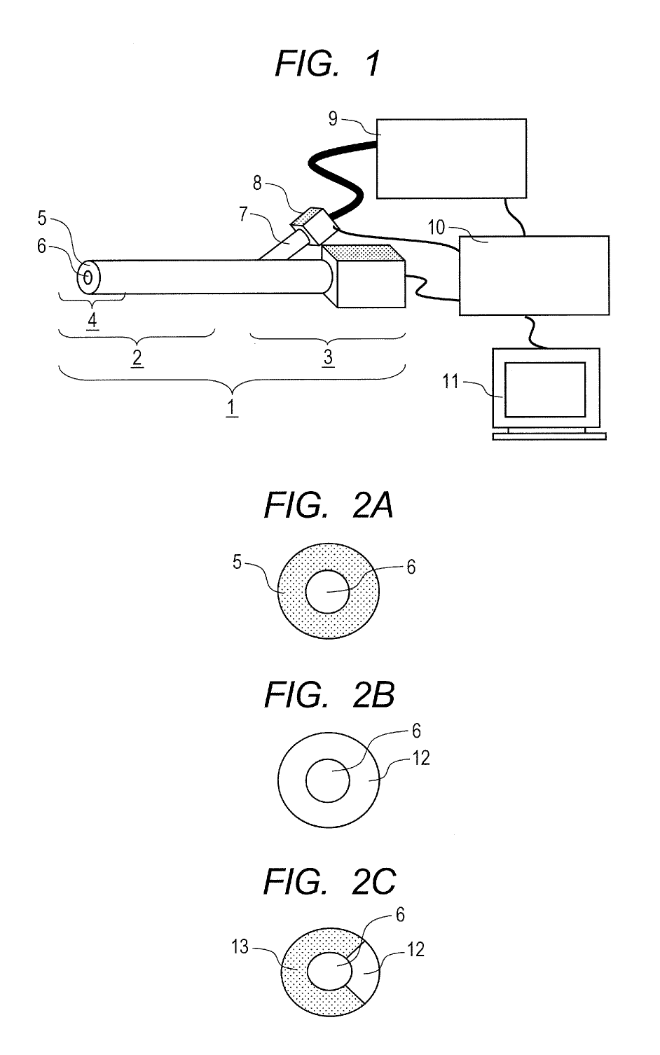

As illustrated in FIG. 1, for example, an endoscopic system in one embodiment of the present invention includes an endoscope 1, a lamp-house 9, an image analysis box 10 and a monitor 11. Although FIG. 1 shows a hard endoscope, a soft endoscope may also be employed as the endoscope 1 of the present invention. The endoscope 1 may be provided with a plurality of objective optical systems or may be a stereoscopic endoscope configured to form a plurality of images by shifting the eyepoint using a monocular objective optical system.

The endoscope 1 roughly includes an insertion section 2 and a grip section 3. The insertion section 2 is to be inserted into a test subject and has a distal end 4 for observation, provided with at least an illumination light exit 5 and an objective optical system 6.

The illumination light exit 5 emits illumination light for observation of the inside of a test subject. The illumination light may be emitted from a light emitting diode (LED) or the like that is provided near the illumination light exit 5 at a part of the distal end 4 of the endoscope, or may be light that is guided from a lamp in the lamp-house 9 to the illumination light exit 5 at the distal end 4 of the endoscope via an optical fiber or a lens as shown in FIG. 1.

The objective optical system 6 includes a light-receiving element such as a CCD provided inside the endoscope 1, and a converter. When receiving reflected light of the illumination light emitted from the illumination light exit 5 at the distal end 4 of the endoscope, the received light forms an image on the light-receiving element, and an observed image is converted into an electric signal by the converter.

The grip section 3 is located outside the test subject, and an observer or an assistant of the observation operation grips the grip section 3 by their hands or using a device for operation. The endoscope of the present embodiment that is of a type configured to guide illumination light via an optical fiber is provided with an illumination light entrance 7 at the grip section 3, and the optical fiber passes through the inside of the endoscope 1 from the illumination light entrance 7 to the illumination light exit 5. In order to convey a larger amount of illumination light to the distal end of the endoscope, the optical fiber preferably is a bundle of about 10,000 thin fibers. In the present embodiment, the system further includes a shutter 8 as illuminance distribution changing means at the illumination light entrance 7, and the shutter 8 is configured to change the illuminating region so as to change the illuminance distribution.

The illumination light used is preferably white light because it enables correct understanding of a reflectance spectrum of a part to be observed in the entire visible wavelengths band. In the present embodiment, a lamp as a light source is provided in the lamp-house 9. Exemplary white lamps used typically include a xenon lamp and a halogen lamp, among which a xenon lamp free from extreme variations in intensity from a short wavelength to a long wavelength is preferably used. A lamp with a specific wavelength may be used depending on their purposes.

An image observed at the distal end 4 of the endoscope is transmitted internally in the endoscope, and is further transmitted to the image analysis box 10 via a signal cable.

The image analysis box 10 analyzes the formed image. The image analysis box 10 of the present embodiment includes: regular reflection light detection means configured to detect blown-out highlights due to regular reflection or the like in the formed image; and brightness determination means configured to determine whether the brightness at an observed region is good for observation or not. The image analysis box 10 is further configured to issue an instruction to adjust the illuminance distribution by the illuminance distribution change means and to adjust illumination light intensity in accordance with a result of such detection and/or determination.

The monitor 11 displays an image to allow an observer to externally observe an image formed by the endoscope. The monitor 11 may be connected to the image analysis box 10 via a video processor to perform image processing such as image correction, or may be directly connected to the image analysis box 10. The video processor may double as the image analysis box 10.

The illuminance distribution in the present invention refers to density distribution of light beams in terms of geometrical optics. This is a concept covering both of expansion of illumination light and intensity of illumination light. Since to change the position of a light source as described in Japanese Patent Application Laid-Open No. 2009-276545 means just the movement of the position and not a change of the density of light beams, such a change is not a change of the illuminance distribution.

The illuminance can be changed by changing the light-emission region of the illumination light or by providing the light-emission region with brightness gradient. Among them, brightness gradient provided in the light-emission region is especially preferable because it facilitates creation of any illuminance distribution and creation of a desired regular reflection state and such a diffused reflection state.

In a specific embodiment, a liquid crystal shutter made up of fine pixels or a neutral density filter (ND filter) may be disposed at the position of the shutter 8 of FIG. 1 for operation. This enables partial light-shielding of the optical fiber bundle, which then changes the illuminating region at the illumination light exit 5 of the distal end 4 of the endoscope and so changes the illuminance distribution of the illumination light emitted from the endoscope distal end. This neutral density filter may be a variable ND capable of changing the amount of darkening so as to provide brightness gradient in the light-emission region. In a more preferable configuration, in addition to the shutter, voltage applied to the lamp in the lamp-house 9 may be changed so as to enable independent control of the brightness of illumination light because such a configuration can broaden the selectable range of the illuminance distribution. FIGS. 2A, 2b and 2C show the illumination light exit at the endoscope distal end and the illumination states as examples of changed illuminance distribution. FIGS. 2A, 2b and 2C show the distal end 4 of the endoscope, where FIG. 2A shows the case where the entire region of the optical fibers located at the illumination light exit 5 is turned off, FIG. 2B shows the case where the entire region is turned on and FIG. 2C shows the case of partially turning-on. Appropriate adjustment of an illuminating region 12 and a non-illuminating region 13 as in FIG. 2C enables desired illuminance distribution while reducing regular reflection.

In another configuration where a light source is provided at the distal end of the endoscope so that the light source doubles as the illumination light exit 5, the light source may be configured to partially turn off or to change the illuminance and the orientation, for example, thus changing the illuminance distribution.

The endoscopic system of the present embodiment is further provided with brightness determination means configured to determine whether the brightness of an image obtained gets dark or not after the change of the illuminance distribution. When the brightness determination means determines that the brightness of the image is not within an appropriate range, illumination light intensity changing means increases the intensity of the illumination light, whereby blown-out highlights due to regular reflection or the like can be reduced while keeping the illumination state of the observation part at brightness enabling favorable observation.

When the endoscope 1 is a stereoscopic endoscope, the endoscopic system may include a circuit to generate a 3D-image from information obtained by the plurality of objective optical systems or by the monocular objective optical system. In the configuration including a plurality of objective optical systems, illuminance distribution that is optimized for one of the objective optical systems may not be appropriate for another objective optical system. Then, optimized illuminance distribution may be found beforehand for each of the objective optical systems, which may be stored in a memory. The objective optical systems may be configured so that their image-forming timings can be individually controlled, and the image-forming timing of each objective optical system is made in synchronization with changing to the optimized illuminance distribution for its image-forming optical system while referring to the memory. In this way, an image formed by any objective optical system can have reduced degree of blown-out highlights. At this time, illumination is set at the illuminance distribution optimized for one of the objective optical systems in a basic state, and the illuminance distribution may be changed before image forming by another objective optical system so as to be suitable to the objective optical system.

### Example 1

In the endoscopic system used in Example 1, the endoscope used was a commercially available hard endoscope having φ10 mm and 250 mm in length. The CCD used had 1.23 millions pixels. The illumination in the lamp-house was a xenon lamp of 300 w.

The illuminance distribution was changed by a liquid crystal shutter provided at the part of the shutter 8, and additionally the brightness of the lamp in the lamp-house was controlled independently by changing the voltage applied thereto.

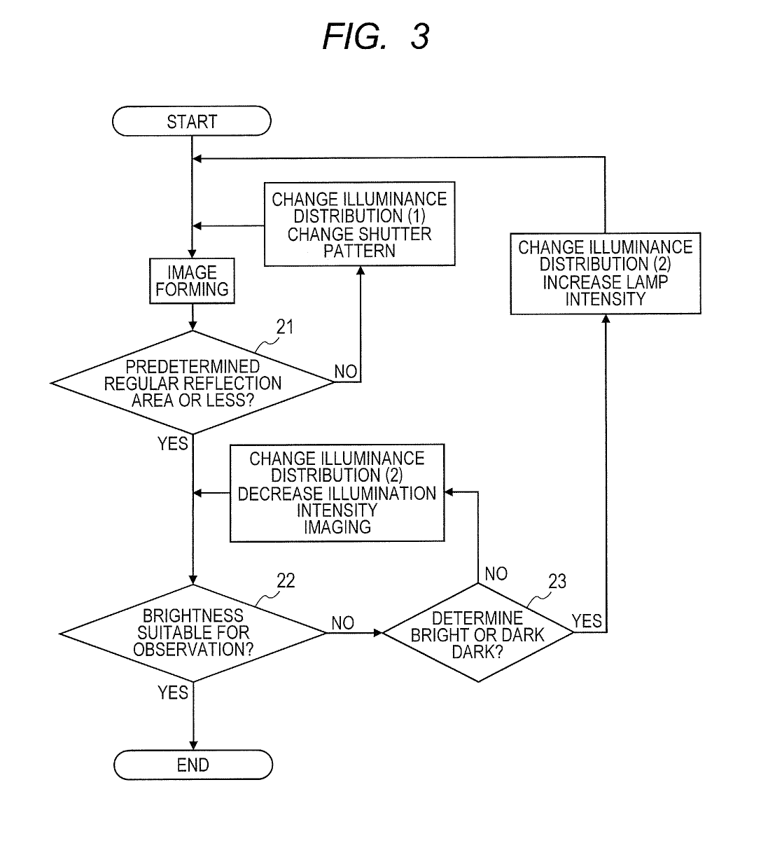

FIG. 3 is a flowchart to change the illuminance distribution in Example 1.

Firstly at the initial illumination state, an image inside a test subject is formed. The formed image undergoes the detection of blown-out highlights due to regular reflection or the like at Step 21 to determine a regular reflection area. When the regular reflection area is a predetermined area or more, illuminance distribution is changed repeatedly until the area becomes the predetermined area or less by adjusting the liquid crystal shutter at the part of the shutter 8. When the regular reflection area becomes the predetermined area or less, determination is made at Step 22 as to whether the brightness of the formed image is within an appropriate range or not. When the brightness is within the appropriate range, the procedure ends. When the brightness is not within the appropriate range, the procedure proceeds to Step 23 to determine whether the formed image is bright or dark. When the brightness is a lower limit of the appropriate range or less, the lamp intensity is increased to increase the illumination intensity. Such an increase of the illumination intensity, however, may increase the regulation reflection area, and so when the illumination intensity is increased, the procedure returns to Step 21 to determine the regular reflection area of the formed image. When the formed image is determined as too bright at Step 23, then the illumination intensity is decreased to adjust the illuminance distribution, thus changing the brightness in the appropriate range. At this time, since the regular reflection area does not increase, Step 22 is performed again so as to determine whether the formed image is within the appropriate range for brightness or not, and thereafter the procedure ends.

Example 1 includes two steps to determine whether illuminance distribution is to be changed or not, i.e., including the step of determining the regular reflectance area and the step of determining brightness. However, these two steps may not be separated depending on algorithms used.

In the flowchart of FIG. 3, the determinations are made as follows. That is, the image analysis box 10 counts the number of pixels of the CCD having signal intensity within a predetermined range and divides the number by the total number of pixels of the CCD, thus measuring a covering area ratio.

Specifically at Step 21 to determine the regular reflection area of the formed image, pixels having signal intensity of 90% or more with reference to 100% that is the upper limit of the signal intensity that the CCD can receive are determined as pixels generating regular reflections detected there, and then when the number of the pixels where such regular reflection is detected is 10% or more of the total number of pixels, it is determined that regular reflection is detected in the formed image. Herein the range (called a first range in the present invention) of signal intensity to determine that the regular reflection is detected at the pixels or the predetermined number of pixels to determine that regular reflection is detected in the formed image is not limited to these numeric values, and they may be any numeric values suitable for the purpose of observation.

At Step 22 to determine whether the formed image has brightness in an appropriate range or not, pixels having signal intensity of 20% or more and 80% or less with reference to 100% that is the upper limit of the signal intensity that the CCD can detect are determined as pixels having brightness within an appropriate range, and then when the number of pixels having brightness in an appropriate range is 70% or more of the total number of pixels, it is determined that the formed image has brightness in an appropriate range. Herein, the range (called a second range in the present invention) of signal intensity to determine that each pixel has brightness in an appropriate range or the predetermined number of pixels to determine that the formed image has brightness in an appropriate range is not limited to these numeric values, and they may be any numeric values suitable for the purpose of observation.

At Step 23 to determine whether the formed image is bright or dark, comparison is made between the number of pixels having the signal intensity of 20% or lower of the upper limit of the signal intensity that the CCD can detect and the number of pixels having the signal intensity of 80% or more thereof. When the number of pixels having the signal intensity of 20% or less is more, the formed image is determined as dark. On the other hand, when the number of pixels having the signal intensity of 80% or more is more, the formed image is determined as bright. Herein, the ranges of the signal intensity (called a third range and a fourth range in the present invention) for comparison of the number of pixels to determine brightness of the image and the predetermined number of pixels as a reference to determine brightness of the image are not limited to these numeric values, and they may be any numeric values suitable for the purpose of observation. The number of pixels in the third range and the number of pixels in the fourth range may be the same, and in that case, the illumination intensity may be increased and the procedure may be performed again from Step 21 to determine the regular reflection area.

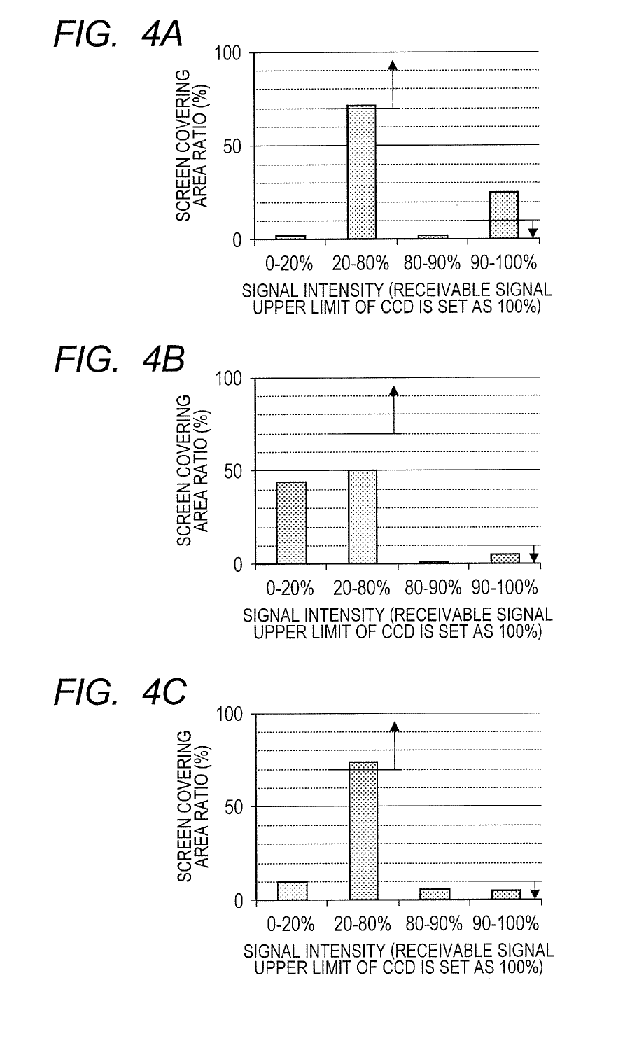

FIGS. 4A, 4B and 4C show the regular reflection area and the area in a brightness appropriate range (the number of pixels for each signal intensity range) in Example 1. FIG. 4A shows a result of the initial image forming, FIG. 4B shows a result of image forming when the illuminance distribution has been changed by the liquid crystal shutter filter and FIG. 4C shows a result of image forming when the illuminance distribution has been changed to brightness good for observation by further changing the intensity of the lamp. FIG. 4A shows the result of image forming before performing the procedure of the flowchart of FIG. 3. As a result of Step 21 to determine the regular reflection area of the flowchart of FIG. 3, the number of pixels where the regular reflection was detected was 25% of the total number of pixels, and so the illuminance distribution was changed by adjusting the liquid crystal shutter. As a result, as shown in FIG. 4B, the number of pixels where the regular reflection was detected was 5% of the total number of pixels. Then, the determination of this result at Step 22 to determine whether brightness is within an appropriate range or not showed that the number of pixels in the range of 20 to 80% of the signal intensity as the appropriate range of the brightness was 50% of the total number of pixels, and so it was determined as beyond the appropriate range. Since the number of the pixels having signal intensity of 20% or less was 40% and the number of pixels having signal intensity of 80% or more was 1%, the formed image was determined as dark at the brightness determination Step 23 of the flowchart of FIG. 3. Then, the illumination intensity of the light was increased. Finally, the illuminance distribution and the illumination intensity were adjusted so as to satisfy the requirements at Step 21 to determine the regular reflection area in the formed image and at Step 22 to determine whether the brightness of the formed image was within an appropriate range or not, whereby an image with less regular reflection and having brightness within an appropriate range was successfully formed as shown in FIG. 4C.

### Comparative Example 1

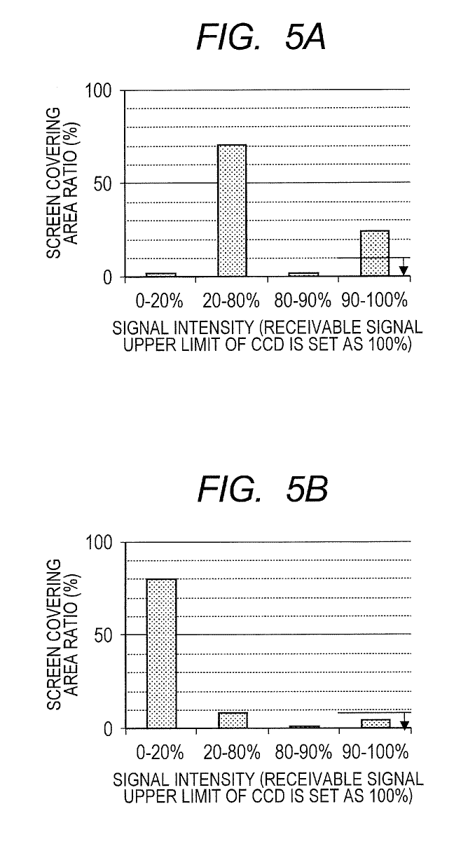

FIG. 5 shows the result of Comparative Example 1, in which regular reflection was reduced by changing the illumination position as in Japanese Patent Application Laid-Open No. 2009-276545. This example was different from Example 1 in that regular reflection was reduced by changing the illumination position without changing the illuminance distribution. FIG. 5A shows a result of the initial image forming, and FIG. 5B shows a result of image forming when the illumination position was changed to reduce regular reflection. As shown in FIG. 5B, although the regular reflection corresponding to the signal intensity of 90% or more was reduced, at the same time a central part to be observed in the screen became dark, thus causing difficulty in observation.

### Example 2

Example 2 was the same as Example 1 except that an image was formed using an endoscope configured to form a plurality of images at different positions, illuminance distribution was changed appropriately for each image-forming position as in Example 1 and the image-forming timing at each image-forming position and the illumination timing at the image-forming position with appropriate illuminance distribution were in synchronization with each other so as to turn on the illumination with the appropriate illuminance distribution at the right image-forming timing. Specifically, a stereoscopic endoscope including two objective optical systems 6 at the distal end as shown in FIG. 6 was used.

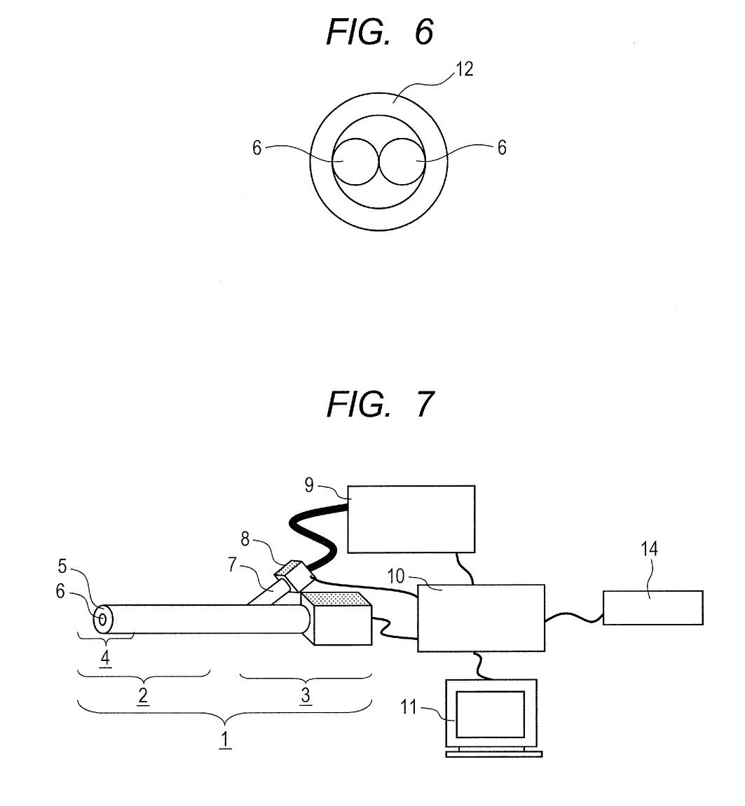

A stereoscopic endoscope typically includes an objective optical system for right-eye image acquisition to form an image for the right eye and an optical system for left-eye image acquisition to form an image for the left eye at mutually shifted positions, thus enabling stereoscopic viewing. The image was formed at the rate of 60 frames/sec., and 30 frames were given to form an image by the objective optical system for right eye and 30 frames similarly were given to form an image by the objective optical system for left eye, and image-formation was alternately performed between the systems for right eye and for left eye. Illumination also was made in synchronization with the objective optical system for right eye and the objective optical system for left eye, and switching between the illumination for right eye and the illumination for left eye was performed 60 times/sec. Illuminance distribution optimized for each objective optical system was found beforehand, which was stored in a memory 14 of FIG. 7. Then, before image-forming, the memory 14 was referred to, and the illumination was turned on so as to have the optimized illuminance distribution suitable for the image forming optical system in synchronization therewith. As a result, the number of loops of algorithm performed was similar to that of the monocular system, thus changing to the optimum region, and similarly to the case of the monocular system with less blown-out highlights, a favorable image was displayed.

### Comparative Example 2

Comparative Example 2 used the same endoscope as that of Example 2. However, optimization of illuminance distribution was performed by applying the method of the present invention to both of images formed by the left and right objective optical systems, and these illuminance distributions obtained were converged into one illuminance distribution for use.

Since there is a difference in the degree of blown-off highlights between the image formed by the objective optical system for left eye and the image formed by the objective optical system for right eye, the application of the algorithm according to the method of the present invention was applied thereto, failing to converge into a state free from blown-off highlights, which are required for each of the images acquired by the left and right objective optical systems. Especially, the amount of remaining blown-off highlights increased in one eye compared with Example 2.

### Comparative Example 3

In Comparative Example 3, illuminance distribution optimized for the objective optical system for right eye was found beforehand, and without changing such illuminance distribution, images were formed alternately using the objective optical system for right eye and the objective optical system for left eye in a similar manner to Example 2.

Then, the image formed by the objective optical system for right eye had reduced blown-off highlights. However, since illumination was not adjusted suitable for the presence or not of blown-off highlights in the image formation by the objective optical system for left eye, blown-off highlights were left in the displayed image for left eye.

According to the present invention, glare due to regular reflection can be reduced, and so favorable observation with an endoscope is enabled.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Applications No. 2012-135579, filed on Jun. 15, 2012, and No. 2013-058690, filed on Mar. 21, 2013, which is are hereby incorporated by reference herein in their entirety.

What is claimed is:

1. An endoscopic system comprising an endoscope including: a light source of illumination light that illuminates inside of a test subject; an illumination light exit, from which the illumination light is emitted; and an objective optical system configured to form an image of the inside of the test body illuminated by the illumination light, wherein the endoscopic system further comprises:
regular reflection light detection means configured to detect blown-off highlights in an image formed by the objective optical system; and
illuminance distribution changing means configured to change illuminance distribution of the illumination light when the regular reflection light detection means detects blown-off highlights.

2. The endoscopic system according to claim 1, further comprising:
brightness determination means configured to determine whether brightness of an image formed by the objective optical system is within an appropriate range for observation or not; and
illumination light intensity changing means configured to, when it is determined that brightness is not within the appropriate range, change intensity of the illumination light.

3. The endoscopic system according to claim 1, wherein the illuminance distribution changing means changes a light-emission region of the illumination light emitted from the illumination light exit or it provides the illumination light emitted from the illumination light exit with brightness gradient.

4. The endoscopic system according to claim 1, wherein the illuminance distribution changing means comprises a liquid crystal shutter or a neutral density filter.

5. The endoscopic system according to claim 1, wherein the regular reflection light detection means determines that blown-off highlights are detected at a pixel having signal intensity in a first range.

6. The endoscopic system according to claim 5, wherein the first range is 90% or more of signal intensity that the objective optical system can detect.

7. The endoscopic system according to claim 5, wherein the illuminance distribution changing means changes illuminance distribution of illumination light when the number of pixels having signal intensity in the first range is a predetermined value or more, and/or illumination light intensity changing means changes intensity of illumination light when the number of pixels having signal intensity in a second range is not a predetermined value or more.

8. The endoscopic system according to claim 5, wherein the illuminance distribution changing means changes illuminance distribution of illumination light when the number of pixels having signal intensity in the first range is 10% or more of a total number of pixels.

9. The endoscopic system according to claim 7, wherein the second range is 20% or more and 80% or less of signal intensity that the objective optical system can detect.

10. The endoscopic system according to claim 9, wherein the illumination light intensity changing means changes intensity of illumination light when the number of pixels having signal intensity in the second range is not 70% or more of a total number of pixels.

11. The endoscopic system according to claim 2, wherein the illumination light intensity changing means
compares the number of pixels having signal intensity in a third range and the number of pixels having signal intensity in a fourth range, the fourth range being higher than the third range,
increases intensity of illumination light when the number of pixels having signal intensity in the third range is more than and is equal to the number of pixels having signal intensity in the fourth range, and
decreases intensity of illumination light when the number of pixels having signal intensity in the third range is less than the number of pixels having signal intensity in the fourth range.

12. The endoscopic system according to claim 11, wherein the third range is 20% or less of signal intensity that the objective optical system can detect, and/or the fourth range is 80% or more of signal intensity that the objective optical system can detect.

13. The endoscopic system according to claim 1, further comprising a plurality of objective optical systems, and a memory that stores illuminance distribution found beforehand enabling optimization of each of the objective optical systems.

14. The endoscopic system according to claim 13, wherein the plurality of objective optical systems can be controlled independently for timing of image formation.

15. The endoscopic system according to claim 13, further comprising a circuit that generates a 3D-image based on information obtained by the plurality of objective optical systems.

16. An image formation method by an endoscopic system, comprising the steps of:
detecting blown-off highlights in a formed image; and
changing illuminance distribution of illumination light when blown-off highlights are detected.

17. The image formation method according to claim 16, further comprising:
determining whether brightness of the image formed is within an appropriate range for observation or not;
when it is determined that brightness is not within the appropriate range, changing intensity of the illumination light.

18. The image formation method according to claim 16 by an endoscopic system including a plurality of objective optical systems, and a memory that stores illuminance distribution found beforehand enabling optimization of each of the objective optical systems, comprising the steps of:
forming images by the plurality of objective optical systems at different timings; and
in the image-forming step, changing illuminance distribution while referring to the memory so as to be in synchronization with timing when each objective optical system forms an image.

19. The image formation method according to claim 18, wherein
the plurality of objective optical systems include an objective optical system to acquire a right-eye image and an objective optical system to acquire a left-eye image, and
the illuminance distribution is changed for any one of illumination to acquire a right-eye image and illumination to acquire a left-eye image.

20. The image formation method according to claim 19, wherein acquisition of a right-eye image by the objective optical system to acquire a right-eye image and acquisition of a left-eye image by the objective optical system to acquire a left-eye image are performed alternately.
